(19) United States
(12) Patent Application Publication
(10) Pub. No.: US 2012/0022292 A1
(43) Pub. Date: Jan. 26, 2012

(54) METHOD FOR PREPARING EPLIVANSERIN HEMIFUMARATE

(75) Inventors: Eric Garcia, Paris (FR); Christian Hoff, Paris (FR)

(73) Assignee: SANOFI-AVENTIS, Paris (FR)

(21) Appl. No.: 13/128,756

(22) PCT Filed: Nov. 10, 2009
(86) PCT No.: PCT/FR2009/052160

§ 371 Date: Oct. 12, 2011

(30) Foreign Application Priority Data
Nov. 14, 2008 (FR) 08/06373

(51) Int. Cl. C07C 249/12

(52) U.S. Cl. 564/256

(57) ABSTRACT

Method for preparing eplivanserin hemifumarate.

The present invention relates to a novel process for the preparation of eplivanserin (1-(2-fluorophenyl)-3-(4-hydroxyphenyl)prop-2-en-1-one O-[2-(dimethylamino)ethyl]oxime) hemifumarate of formula:

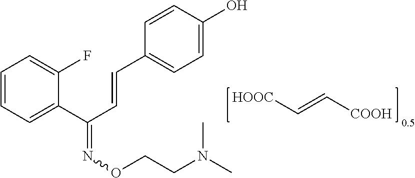

Eplivanserin can exist in the form of two isomers, according to the Z or E configuration of the C═N double bond, the C═C double bond being of E configuration. During its synthesis, eplivanserin is obtained in the form of a mixture of the two isomers (Z and E) of the C═N double bond of the oxime functional group. This mixture will be referred to subsequently as “eplivanserin base”. As the Z isomer of eplivanserin is more particularly advantageous, it is therefore necessary to efficiently separate the two isomers. A novel preparation process which makes it possible to isolate and crystallize the desired eplivanserin isomer is therefore particularly expected.

The processes for the isomerization of oximes known from the state of the art generally require catalysts of the strong acid or Lewis acid type (see, for example, the documents WO98/32758, WO03/033488 or EP 0 435 687) or light radiation (document WO 2006/067092). Surprisingly, the preparation process according to the present invention does not require such additives. Eplivanserin and its salts, and their method of preparation, are described in document EP 0 373 998. However, the process described does not make it possible to satisfactorily separate the two Z and E isomers, that is to say so as to obtain less than 0.5% of the undesirable E isomer, while avoiding nucleation (formation of new crystals) of this E isomer at the end of the reaction. Furthermore, this process cannot be satisfactorily transferred to the industrial scale. The present invention makes it possible to overcome such disadvantages while furthermore resulting in a significant improvement in the yield.

Eplivanserin hemifumarate is described in particular in the document EP 0 373 998 and is known to be a serotonin 5HT_{2A} receptor antagonist (Journal of Pharmacology and Experimental Therapeutics (1992), vol. 262 (2), pp. 759-68). It is particularly useful in the treatment of sleep disorders (Neuropsychopharmacology (1999), vol. 21 (3), pp, 455-466, and document WO2007/020337).

The synthetic process according to the present invention is characterized in that the eplivanserin base is isomerized and crystallized, by the action of fumaric acid, in the presence of a polar solvent, the boiling point of which is greater than 100° C., or of a mixture of polar solvents, the boiling point of which is greater than 100° C., by the following stages:
i) heating the eplivanserin base in the presence of fumaric acid and then cooling,
ii) seeding,
iii) lowering the temperature via stationary phases, 20
iv) filtering and washing.

In comparison with the document EP 0 373 998, this novel process makes it possible to efficiently isolate the Z isomer from the E isomer, while preventing the reformation of the latter by nucleation, and also reduces the stages for the isolation of the finished product, thereby resulting in a significant saving in time. A doubling of the theoretical yield (100% instead of 50%) is also observed, the process being compatible with large scale industrial production.

The eplivanserin base is isomerized and crystallized in the presence of an acid, such as fumaric acid, and in a polar solvent, the boiling point of which is greater than 100° C., such as isobutanol (2-butanol), n-butanol or n-pentanol, or in a mixture of polar solvents, the boiling point of which is greater than 100° C. Mention may in particular be made, as example of such a mixture, of an isobutanol/DMF (dimethylformamide) mixture. According to one embodiment, the solvent is isobutanol.

A boiling point of a polar solvent (or of a mixture of polar solvents) of greater than 100° C. makes possible the isomerization during crystallization and thus makes it possible to develop the optimum conditions necessary for the kinetics of the process.

The mixture is heated, preferably above 100° C., in order to be able to initiate the crystallization at the isomerization equilibrium, and then cooled via stationary phases down to the filtration temperature. For example, when the solvent is isobutanol, the mixture is heated to 105° C., which ensures that the isomerization equilibrium is reached, and then cooled down to 100° C.

Seeding is understood to mean the introduction of crystals of the desired form into the crystallization medium comprising the eplivanserin base. Preferably, seeding will be carried out using (1Z, 2E)-1-(2-fluorophenyl)-3-(4-hydroxyphenyl)prop-2-en-1-one O-[2-(dimethylamino)ethyl]oxime hemifumarate, prepared, for example, according to the document EP 0 373 998.

Polar solvent is understood to mean a solvent which has a high dipole moment, such as water, acetone, ammonia, alcohols. DMSO or DMF. Two categories are distinguished among polar solvents: protic solvents, which can give rise to hydrogen bonds (such as water or alcohols), and aprotic solvents, which cannot form them in the absence of a labile proton (such as DMF or DMSO).

Temperature stationary phases is understood to mean a gradual lowering of the temperature over time, characterized by a pair (T, t): T representing the temperature of the stationary phase and t the duration of the stationary phase. Between two stationary phases, the temperature is lowered, preferably by 5° C. per hour, it being essential for the final temperature not to be less than 70±5° C. The duration t of a stationary phase is preferably between 30 min and 6 hours, in particular between 4 hours and 6 hours. The filtration and the washing can be carried out by any method known to a person skilled in the art.

A product obtained according to the above process which is particularly preferred is 1-(2-fluorophenyl)-3-(4-hydroxyphenyl)prop-2-en-1-one O-[2-(dimethylamino)ethyl]oxime hemifumarate comprising a percentage of (Z, E) isomer of greater than 99.5%.

A product obtained according to the above process which is particularly preferred is 1-(2-fluorophenyl)-3-(4-hydroxyphenyl)prop-2-en-1-one O-[2-(dimethylamino)ethyl]oxime hemifumarate comprising a percentage of (E, E) isomer of less than 0.5%.

The eplivanserin salt obtained can subsequently be purified, for example by a stage of crystallization or by any other method known to a person skilled in the art.

The eplivanserin base can be obtained by the following stages:

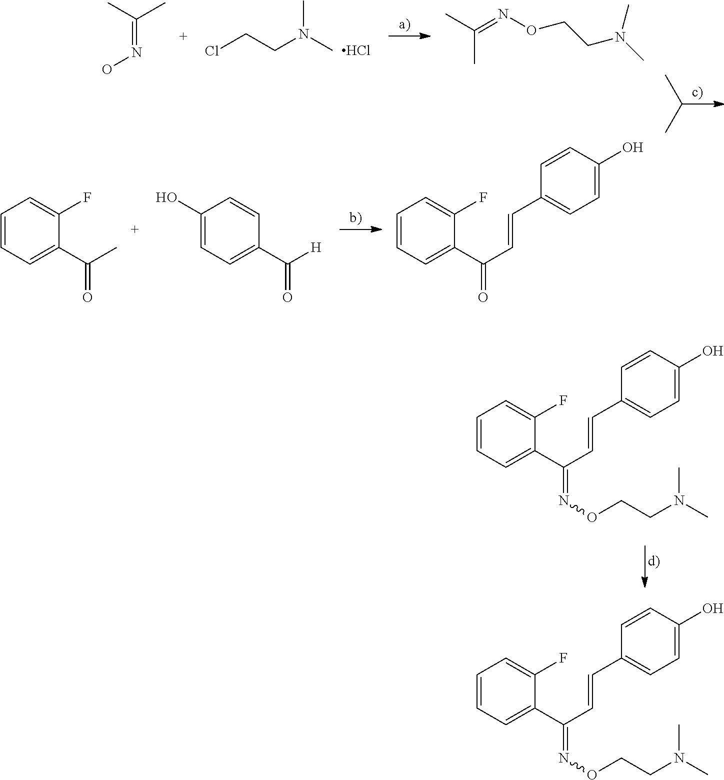

a) acetone oxime is condensed with (2-chloroethyl)dimethylamine in the presence of a base and of a solvent, to form the dimethylaminoacetoxime;
b) 4-hydroxybenzaldehyde is condensed with 2-fluoroacetophenone in a solvent, to form the 2-fluorohydroxychalcone;
c) the 2-fluorohydroxychalcone obtained in stage b) is condensed with the dimethylaminoacetoxime obtained in stage a) in a solvent, to form the eplivanserin base;
d) the eplivanserin base is crystallized from a solvent.

The solvent used in stage d) is toluene.

The eplivanserin base obtained in stage c) is obtained from the 2-fluorohydroxychalcone and the dimethylaminoacetoxime in the presence of a solvent, such as n-butanol.

The 2-fluorohydroxychalcone is obtained from 4-hydroxybenzaldehyde and 2-fluoroacetophenone in a solvent chosen from: ethanolic hydrochloric acid solution, pentanolic hydrochloric acid solution, isopropanolic hydrochloric acid solution or n-butanolic hydrochloric acid solution, at a temperature of less than 30° C. According to one embodiment, the solvent is ethanolic hydrochloric acid solution.

The dimethylaminoacetoxime is obtained from acetone oxime and (2-chloroethyl)dimethylamine in the presence of a base, such as NaOH or KOH, and of a polar solvent, such as THF (tetrahydrofuran). According to one embodiment, the base is KOH. The dimethylacetoxime can be isolated in the form of an oxalate salt or retained in the base form in solution in a THF/MTBE (methyl tert-butyl ether) mixture.

### EXAMPLE 1

### Synthesis of Eplivanserin Hemifumarate

80 kg of 2-fluoroacetophenone and 67 kg of 4-hydroxybenzaldehyde are dissolved in ethanol. An ethanolic hydrochloric acid solution (at least 64 kg) is added to the mixture and then the medium is stirred at a temperature of less than 30° C. for 5 h 15. The medium is cooled and the 2-fluorohydroxychalcone is observed to crystallize, which product is filtered off and washed with toluene, and then dried under vacuum at a temperature of less than 60° C.

Chemical yield starting from 2-fluoroacetophenone: 75±15%.

An aqueous solution of (2-chloroethyl)dimethylamine (CDMA) (246 kg of a 65% w/w solution) is added to a mixture of acetone oxime (162 kg) and potassium hydroxide (160 kg) in THF (600 l). The reaction medium is brought to reflux for at least 5 h. The medium is subsequently diluted with water, an aqueous NaCl solution and an aqueous sodium hydroxide solution and then extracted with MTBE. The organic phase is washed with an NaCl and sodium hydroxide solution, to result in the dimethylaminoacetoxime (DMA acetoxime) in MTBE solution, used as is in the stage of coupling with the 2-fluorohydroxychalcone.

The dimethylaminoacetoxime solution as prepared above (105 kg of dimethylaminoacetoxime, 100% base) is added to a solution of oxalic acid (100 kg) in 1-butanol (600 l). The THF and the MTBE are stripped off with 1-butanol and the oxalate salt obtained is reacted with the 2-fluorohydroxychalcone (90 kg) in the presence of aqueous HCl (202 kg). The mixture is brought to 102° C. for at least 6 hours, the acetone being distilled off as it is formed and being replaced with 1-butanol. At the end of the reaction, the 1-butanol is replaced with water by distillation. The aqueous phase obtained is washed with MTBE, brought back to neutral pH by addition of concentrated ammonium hydroxide and then brought to pH 9-9.5 by addition of a sodium carbonate solution. The solid obtained is then filtered off, washed with water and dried. The crude eplivanserin base mixture is subsequently dissolved in toluene (1580 l) at approximately 95° C. The solution is filtered under hot conditions through a filter medium impregnated with active charcoal and then the solution is cooled, making possible the crystallization of the eplivanserin base, which is filtered off and dried under vacuum at a temperature of less than 70° C.

Chemical yield starting from the 2-fluorohydroxychalcone: 80±15%

The mixture of Z and E isomers (eplivanserin base) (107 kg) and the fumaric acid (19 kg) are dissolved in isobutanol (approximately 1700 l) and the mixture is brought to reflux until dissolution is complete. The medium is kept at reflux for approximately half an hour. The medium is subsequently cooled to 100° C. with a cooling gradient of approximately −10° C./h. Approximately 2% of (1Z,2E)-1-(2-fluorophenyl)-3-(4-hydroxyphenyl)prop-2-en-1-one O-[2-(dimethylamino)ethyl]oxime hemifumarate are introduced into the solution. Isothermal conditions are observed for ½, hour, before cooling the suspension to approximately 90° C. with a cooling gradient of −5° C./h. A stationary phase at 90° C. of 6 h is observed, before again cooling the suspension to 85° C. with a cooling gradient of −5° C./h. A stationary phase at 85° C. of 6 h is observed, before cooling to 82.5° C. with a cooling gradient of −5° C./h. A stationary phase at the temperature of 82.5° C. of 4 h is maintained, before cooling to 80° C. with a cooling gradient of −5° C./h. The temperature is maintained at 80° C. for 4 hours, before cooling to 70° C. with a cooling gradient of −2.5° C./h. The temperature is maintained under isothermal conditions at 70° C. for approximately half an hour, before filtering the suspension. The powder obtained is washed with approximately 170 l of ethanol and then dried at approximately 45° C. under vacuum. There are thus obtained approximately 96 kg of powder formed of (1Z,2E)-1-(2-fluorophenyl)-3-(4-hydroxyphenyl)prop-2-en-1-one O-[2-(dimethylamino)ethyl]oxime hemifumarate comprising less than 0.5% of the (E,E) isomer, i.e. a yield of approximately 77%.

In a comparable manner, it is possible to obtain similar results by using, as polar solvent, for example n-butanol or n-pentanol or, as a mixture of polar solvents, an isobutanol/DMF (95/5) mixture.

1. A process for the preparation of eplivanserin hemifumarate, characterized in that the eplivanserin base is isomerized and crystallized, by the action of fumaric acid, in the presence of a polar solvent, the boiling point of which is greater than 100° C., or of a mixture of polar solvents, the boiling point of which is greater than 100° C., comprising the steps of:
i) heating the eplivanserin base in the presence of fumaric acid and then cooling,
ii) seeding,
iii) lowering the temperature via stationary phases, and
iv) filtering and washing.

2. The process according to claim 1, wherein the polar solvent is isobutanol.

3. The process according to claim 1, wherein the polar solvent is n-butanol.

4. The process according to claim 1, wherein the polar solvent is n-pentanol.

5. The process according to claim 1, wherein the mixture of polar solvents is an isobutanol/DMF mixture.

6. The process according to claim 1, wherein the product obtained is (1Z, 2E)-1-(2-fluorophenyl)-3-(4-hydroxyphenyl)prop-2-en-1-one O-[2-(dimethylamino)ethyl]oxime hemifumarate.

7. The process according to claim 1, wherein step (i) the mixture is heated to 105° C. and then cooled to 100° C.

8. The process according to claim 1, wherein step (ii) seeding is carried out with (1Z, 2E)-1-(2-fluorophenyl)-3-(4-hydroxyphenyl)prop-2-en-1-one O-[2-(dimethylamino)ethyl]oxime hemifumarate.

9. The process according to claim 1, wherein step (iii) or the temperature between two stationary phases is lowered by 5° C. per hour, down to 70° C.

10. The process according to claim 1, wherein step (iii) the duration of a stationary phase is between 30 minutes and 6 hours.

11. The process according to claim 1, wherein step (iii) the duration of a stationary phase is between 4 hours and 6 hours.

12. The process according to claim 1, wherein the eplivanserin base is obtained by the following stages: 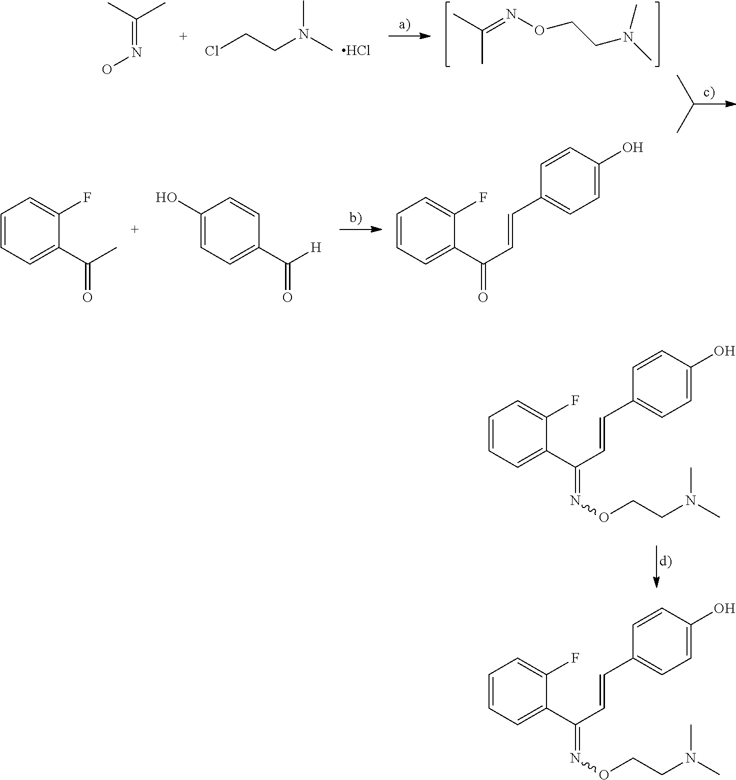
a) acetone oxime is condensed with (2-chloroethyl)dimethylamine in the presence of a base and of a solvent, to form the dimethylaminoacetoxime;
b) 4-hydroxybenzaldehyde is condensed with 2-fluoroacetophenone in a solvent, to form the 2-fluorohydroxychalcone;
the 2-fluorohydroxychalcone obtained in stage b) is condensed with the dimethylaminoacetoxime obtained in stage a) in a solvent, to form the eplivanserin base;
d) the eplivanserin base is crystallized from a solvent.

13. The process according to claim 12, wherein stage a), the base is KOH and the polar solvent is THF.

14. The process according to claim 12, wherein stage b), the solvent is ethanolic hydrochloric acid solution.

15. The process according to claim 12, wherein stage c), the solvent is n-butanol.

16. The process according to claim 12, wherein, in stage d), the solvent is toluene.

17. 1-(2-Fluorophenyl)-3-(4-hydroxyphenyl)prop-2-en-1-one O-[2-(dimethylamino)ethyl]oxime hemifumarate obtained according to the process according to claim 1 and comprising a percentage of the (Z, E) isomer of greater than 99.5%.

18. 1-(2-Fluorophenyl)-3-(4-hydroxyphenyl)prop-2-en-1-one O-[2-(dimethylamino)ethyl]oxime hemifumarate obtained according to the process according to claim 1 and comprising a percentage of the (E, E) isomer of less than 0.5%.
